(19) United States
(12) Patent Application Publication
(10) Pub. No.: US 2004/0215253 A1
(43) Pub. Date: Oct. 28, 2004

(54) Implantable cardiac stimulation device providing atrial accelerated arrhythmia termination electrode configuration selection and method

(76) Inventor: Lisa P. Weinberg, Moorpark, CA (US)

Correspondence Address: PACESETTER, INC., 15900 VALLEY VIEW COURT, SYLMAR, CA, 91392-9221, US

(21) Appl. No.: 10/424,386

(22) Filed: Apr. 24, 2003

(51) Int. Cl. A61N 1/362

(52) U.S. Cl. 607/9; 607/4; 607/14

(57) ABSTRACT

An implantable cardiac stimulation device includes a system for terminating atrial accelerated arrhythmias of the heart. The system includes at least one lead including a plurality of electrodes adapted to define a plurality of atrial accelerated arrhythmia termination electrode configurations and an arrhythmia detector that detects an atrial tachyarrhythmia of the heart. The system further includes an electrode configuration selector that selects one of the plurality of electrode configurations for termination of the detected atrial tachyarrhythmia based upon a measure of at least one predetermined characteristic of the detected atrial accelerated arrhythmia. The system still further includes a therapy circuit that provides atrial tachyarrhythmia termination therapy to the selected electrode configuration.

### FIELD OF THE INVENTION

The present invention generally relates to an implantable cardiac stimulation device. The present invention more particularly relates to such a device having a system which selects an electrode configuration for terminating a detected accelerated atrial arrhythmia based upon a measure of at least one predetermined characteristic of the detected arrhythmia.

### BACKGROUND OF THE INVENTION

Implantable cardiac devices are well known in the art. They may take the form of implantable defibrillators or cardioverters which treat accelerated rhythms of the heart such as fibrillation or implantable pacemakers which maintain the heart rate above a prescribed limit, such as, for example, to treat a bradycardia. Implantable cardiac devices are also known which incorporate both a pacemaker and a defibrillator.

A pacemaker may be considered as a pacing system. The pacing system is comprised of two major components. One component is a pulse generator which generates the pacing stimulation pulses and includes the electronic circuitry and the power cell or battery. The other component is the lead, or leads, which electrically couple the pacemaker to the heart.

Pacemakers deliver pacing pulses to the heart to cause the stimulated heart chamber to contract when the patient's own intrinsic rhythm fails. To this end, pacemakers include sensing circuits that sense cardiac activity for the detection of intrinsic cardiac events such as intrinsic atrial events (P waves) and intrinsic ventricular events (R waves). By monitoring such P waves and/or R waves, the pacemaker circuits are able to determine the intrinsic rhythm of the heart and provide stimulation pacing pulses that force atrial and/or ventricular depolarizations at appropriate times in the cardiac cycle when required to help stabilize the electrical rhythm of the heart.

Pacemakers are described as single-chamber or dual-chamber systems. A single-chamber system stimulates and senses the same chamber of the heart (atrium or ventricle). A dual-chamber system stimulates and/or senses in both chambers of the heart (atrium and ventricle). Dual-chamber systems may typically be programmed to operate in either a dual-chamber mode or a single-chamber mode.

While pacemakers are generally used to regulate heart rhythms, they have also been used to restore a heart to a regular heart rate from an unduly high or accelerated rate known as a tachyarrhythmia. To this end, overdrive pacing has been employed to capture stable accelerated rhythms, known as tachycardias. Because tachycardias are characterized by organized heart chamber activity, the chamber activity may be captured by overdrive pacing. Once the chamber is captured, the pacing rate may then be gradually decreased to return the heart to a normal heart rate. Such therapy has been used for both ventricle tachycardias and atrial tachycardias. Atrial tachycardias may also be referred to as atrial flutter.

Fibrillation is a tachyarrhythmia which is not characterized by organized chamber activity. Quite to the contrary, the activity of a heart chamber in fibrillation is chaotic. In the ventricles, it is so chaotic that any meaningful heart activity to sustain life is absent. Hence, ventricular fibrillation is immediately life threatening. Fortunately, ventricular defibrillators are capable of restoring a heart from ventricular fibrillation to a normal ventricular rate. This is generally accomplished by the defibrillator immediately delivering a rather high output shock to the ventricles with implanted defibrillation electrodes.

Atrial fibrillation can be precipitated from an immediately proceeding atrial tachycardia or can occur suddenly. It results in rapid and chaotic activity of the atria of the heart. The chaotic atrial activity in turn causes the ventricular activity to become rapid and variable. Although it is not left threatening, it is associated with strokes thought to be caused by blood clots forming in areas of stagnant blood flow as a result of prolonged atrial fibrillation. Symptoms of atrial fibrillation may include heart palpitations and dizziness. Atrial defibrillators are known which can deliver a defibrillation shock to the atria for terminating atrial fibrillation. Hence, it is now common for cardiac stimulation devices to include therapies for terminating atrial tachycardias and fibrillation.

It has been found that in some patients, site-specific pacing may help to prevent the occurrence of atrial fibrillation. Such sites may include dual right atrial pacing at the coronary sinus ostium and right atrial appendage, bi-atrial pacing, atrial septum pacing, or single chamber atrial pacing (right or left only). Automatic electrode configuration selection has also been proposed for use in providing such preventative pacing. Automatic electrode configuration selection has also been used in lead impedance testing, used to determine a malfunctioning electrode configuration, and to support the switching to an operable electrode configuration when the impedance is too high. It has also been used to select a best sensing electrode pair configuration. However, there remains a need in the art for a system and method which provide automatic electrode configuration selection to provide the most effective termination of atrial tachyarrhythmias such as atrial tachycardia and atrial fibrillation.

### SUMMARY

What is described herein is a system for terminating atrial accelerated arrhythmias of a heart. The system comprises at least one lead including a plurality of electrodes adapted to define a plurality of atrial accelerated arrhythmia termination electrode configurations, an arrhythmia detector that detects atrial accelerated arrhythmias of the heart, an electrode configuration selector that selects one of the plurality of electrode configurations for termination of a detected atrial accelerated arrhythmia based upon a measure of at least one predetermined characteristic of the detected atrial accelerated arrhythmia, and a therapy circuit that provides atrial accelerated arrhythmia termination therapy to the selected electrode configuration.

The electrode configuration selector may select a most effective electrode configuration for termination of the detected atrial accelerated arrhythmia from among the plurality of electrode configurations. The electrode configuration selector may also maintain a statistical record correlating electrode configuration atrial accelerated arrhythmia termination effectiveness with measures of the predetermined characteristic.

### BRIEF DESCRIPTION OF THE DRAWINGS

Further features and advantages of the present invention may be more readily understood by reference to the following description taken in conjunction with the accompanying drawings, in which:

FIG. 1 is a simplified diagram illustrating an implantable stimulation device embodying the present invention in electrical communication with a patient's heart for delivering atrial accelerated arrhythmia therapy to the patient's heart;

FIG. 2 is a functional block diagram of the implantable stimulation device of FIG. 1;

FIG. 3 is a flow chart describing an overview of the operation of one embodiment of the present invention;

FIG. 4 is a graph illustrating a statistical record correlating atrial accelerated arrhythmia termination success for different pacing electrode configurations and accelerated arrhythmia rates; and

FIG. 5 is another graph showing a similar statistical record for defibrillation electrode configuration.

### DESCRIPTION OF THE PREFERRED EMBODIMENTS

The following description is of the best mode presently contemplated for practicing the invention. This description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

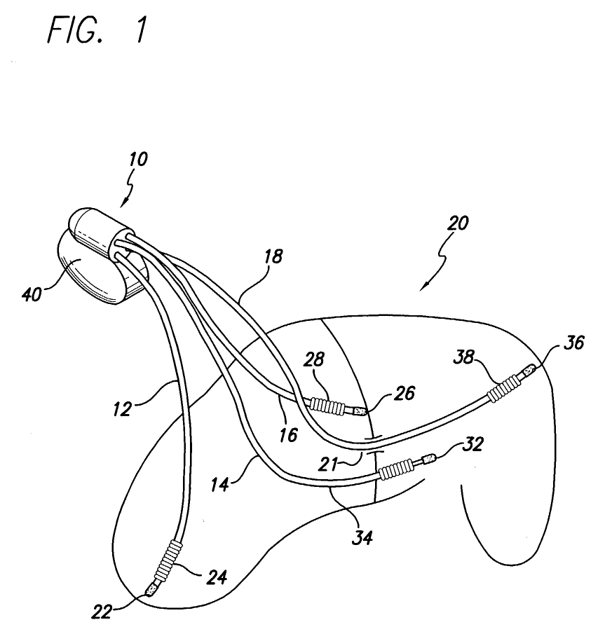

As shown in FIG. 1, there is a stimulation device 10 in electrical communication with a patient's heart 20 by way of four leads, 12, 14, 16 and 18, suitable for delivering atrial accelerated arrhythmia termination pacing stimulation and defibrillation shock therapy. Lead 12 includes a right atrial appendage pacing electrode 22 and defibrillation coil electrode 24. Lead 14 includes a coronary sinus left atrial pacing electrode 32 and defibrillation coil electrode 34. Lead 16 includes a right atrial septal pacing electrode 26 and defibrillation coil electrode 28. Finally, lead 18 includes a left atrial free wall pacing electrode 36 and defibrillation coil electrode 38. The left free wall lead 18 may be placed by piercing the septum 21 and passing the lead 18 through the septum to the left side of the heart. The aforementioned placement of leads is for illustration purposes only, and is not intended as a limitation. It is thus contemplated that multiple leads placed in a variety of locations may be used. Also, as used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. An exemplary coronary sinus lead 14 is designed to receive atrial cardiac signals and to deliver left atrial pacing therapy using left atrial tip electrode 32, and to deliver left atrial shocking therapy using left atrial coil electrode 34.

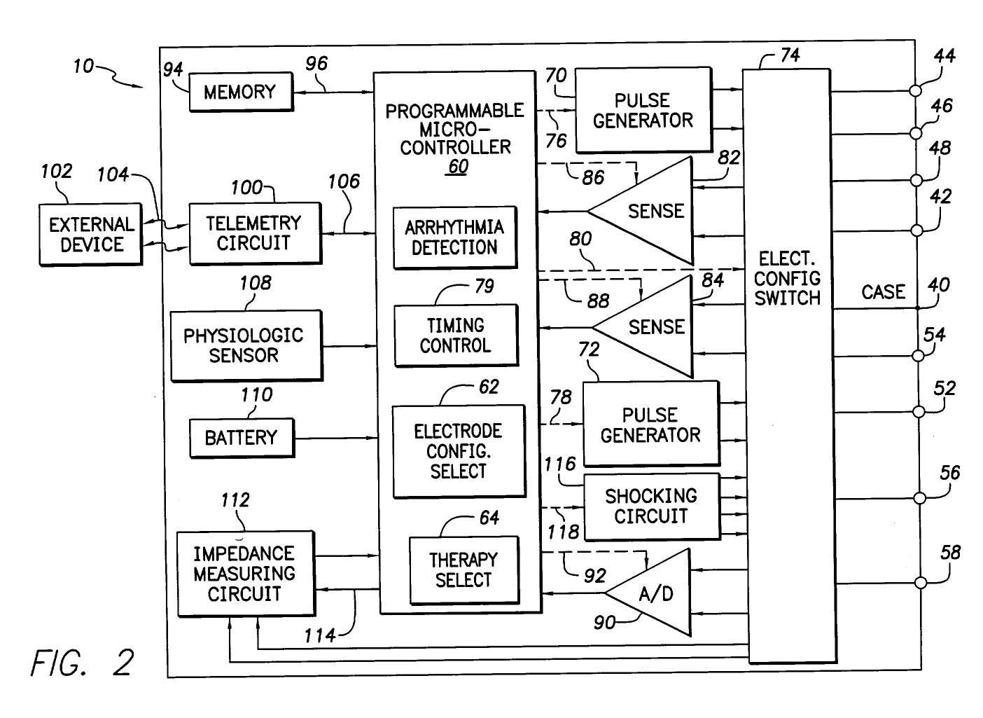

As illustrated in FIG. 2, a simplified block diagram is shown of the implantable stimulation device 10, which is capable of treating both fast and slow atrial arrhythmias with stimulation therapy, including cardioversion, defibrillation, and pacing stimulation. While a particular device is shown, this is for illustration purposes only, and one of skill in the art could readily include further appropriate circuitry in any desired combination to provide a device capable of treating all four heart chamber(s) with cardioversion, defibrillation and pacing stimulation.

The housing 40 for the stimulation device 10, shown schematically in FIG. 2, is often referred to as the “can”, “case” or “case electrode” and may be programmably selected to act as the return electrode for all “unipolar” modes. The housing 40 may further be used as a return electrode alone or in combination with one or more of the coil electrodes, 24, 26, 34 and 38, for shocking purposes. The housing 40 further includes a connector (not shown) having a plurality of terminals, 42, 44, 46, 48, 52, 54, 56, and 58 for connection to electrodes 22, 24, 26, 28, 32, 34, 36, and 38, respectively.

At the core of the stimulation device 10 is a programmable microcontroller 60 that controls the various modes of stimulation therapy. As is well known in the art, the microcontroller 60 typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by a program code stored in a designated block of memory. The details of the design and operation of the microcontroller 60 are not critical to the present invention. Rather, any suitable microcontroller 60 may be used that carries out the functions described herein. The use of microprocessor-based control circuits for performing timing and data analysis functions are well known in the art.

As shown in FIG. 2, atrial pulse generators 70 and 72 generate pacing stimulation pulses for delivery to any one or combination of pacing electrodes 22, 26, 32 and 36 via an electrode configuration switch 74. The pulse generators, 70 and 72, are controlled by the microcontroller 60 via appropriate control signals, 76 and 78, respectively, to trigger or inhibit the stimulation pulses.

The microcontroller 60 further includes timing control circuitry 79 which is used to control the timing of stimulation pulses (e.g., pacing rate, or atrial interconduction (A-A) delay, etc.) as well as to keep track of the timing of refractory periods, blanking intervals, noise detection windows, alert intervals, marker channel timing, etc., which is well known in the art.

The switch 74 includes a plurality of switches for connecting the desired electrodes to the appropriate I/O circuits, thereby providing complete electrode programmability. Accordingly, the switch 74, in response to a control signal 80 from the microcontroller 60, determines the polarity of the stimulation pulses (e.g., unipolar, bipolar, combipolar, etc.) by selectively closing the appropriate combination of switches (not shown) as is known in the art.

Atrial sensing circuits 82 and 84 may also be selectively coupled to any one or combination of electrodes 22, 24, 26, 28, 32, 34, 36, and 38, through the switch 74 for detecting the presence of cardiac activity in each of the atria of the heart. Accordingly, the switch 74 determines the “sensing polarity” of the cardiac signal by selectively closing the appropriate switches, as is also known in the art. In this way, the clinician may program the sensing polarity independent of the stimulation polarity.

Each sensing circuit, 82 and 84, preferably employs one or more low power, precision amplifiers with programmable gain and/or automatic gain control, bandpass filtering, and a threshold detection circuit, as known in the art, to selectively sense the cardiac signal of interest. The automatic gain control enables the device 10 to deal effectively with the difficult problem of sensing low amplitude atrial signals characteristic of atrial fibrillation. The outputs of the sensing circuits, 82 and 84, are connected to the microcontroller 60 which, in turn, are able to trigger or inhibit the pulse generators, 70 and 72, in a demand fashion in response to the absence or presence of cardiac activity in the appropriate chambers of the heart.

For arrhythmia detection, the device 10 utilizes one or both of the atrial sensing circuits, 82 and 84, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, and depolarization signals associated with fibrillation which are sometimes referred to as “atrial Fib-waves”) are then classified by the microcontroller 60 by comparing them to a predefined rate zone limit (i.e., bradycardia, normal, low rate AT, high rate AT, and atrial fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). The atrial rates, as will be seen hereinafter, may be used for selection of a most effective atrial tachyarrhythmia termination electrode configuration.

Cardiac signals are also applied to the inputs of an analog-to-digital (A/D) data acquisition system 90. The data acquisition system 90 is configured to acquire intracardiac electrogram signals, convert the raw analog data into a digital signal, and store the digital signals for later processing and/or telemetric transmission to an external device 102. The data acquisition system 90 may be through the switch 74 to sample atrial signals across any pair of desired electrodes.

The microcontroller 60 is further coupled to a memory 94 by a suitable data/address bus 96, wherein the programmable operating parameters used by the microcontroller 60 are stored and modified, as required, in order to customize the operation of the stimulation device 10 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 20 within each respective therapy.

Advantageously, the operating parameters of the implantable device 10 may be non-invasively programmed into the memory 94 through a telemetry circuit 100 in telemetric communication with the external device 102, such as a programmer, transtelephonic transceiver, or a diagnostic system analyzer. The telemetry circuit 100 is activated by the microcontroller by a control signal 106. The telemetry circuit 100 advantageously allows intracardiac electrograms and status information relating to the operation of the device 10 (as contained in the microcontroller 60 or memory 94) to be sent to the external device 102 through an established communication link 104.

In the preferred embodiment, the stimulation device 10 further includes a physiologic sensor 108, commonly referred to as a “rate-responsive” sensor because it is typically used to adjust pacing stimulation rate according to the exercise state of the patient. Accordingly, the microcontroller 60 responds by adjusting the various pacing parameters (such as rate, A-A Delay, etc.) at which the atrial pulse generators, 70 and 72, generate stimulation pulses.

The stimulation device additionally includes a battery 110 which provides operating power to all of the circuits shown in FIG. 2. For the stimulation device 10, which employs shocking therapy, the battery 110 must be capable of operating at low current drains for long periods of time and then be capable of providing high-current pulses (for capacitor charging) when the patient requires a shock pulse. The battery 110 must also have a predictable discharge characteristic so that elective replacement time can be detected. Accordingly, the device 10 preferably employs lithium/silver vanadium oxide batteries, as is true for most (if not all) current devices.

As further shown in FIG. 2, the device 10 is shown as having an impedance measuring circuit 112 which is enabled by the microcontroller 60 via a control signal 114. The impedance measuring circuit 112 is not critical to the present invention and is shown for only completeness.

When the stimulation device 10 is to apply an appropriate electrical shock therapy to the heart aimed at terminating an atrial accelerated detected arrhythmia, the microcontroller 60 further controls a shocking circuit 116 by way of a control signal 118. The shocking circuit 116 generates shocking pulses of low (up to 0.5 joules), moderate (0.5-10 joules), or high energy (11 to 40 joules), as controlled by the microcontroller 60. Such shocking pulses are applied to the patient's heart 20 through at least two shocking electrodes, and as shown in this embodiment, selected from coil electrodes 24, 28, 34, and 38. As noted above, the housing 40 may act as an active electrode in combination with any one of the coil electrodes or combination of the coil electrodes. Atrial defibrillation shocks are generally considered to be of low to moderate energy level (so as to minimize pain felt by the patient), and are preferably synchronized with an R-wave by circuitry not shown in a manner well known in the art.

Now that the device 10 has been generally described, this description shall now turn to more particular aspects of the present invention. As previously mentioned, in accordance with the present invention, the device, upon detection of an atrial tachyarrhythmia, selects one of a plurality of arrhythmia termination electrode configurations. The selection of the selected electrode configuration is preferably based upon the electrode configuration which will most effectively terminate the arrhythmia as characterized by the measure of at least one predetermined characteristic in the detected arrhythmia. Preferably, the predetermined characteristic is atrial rate.

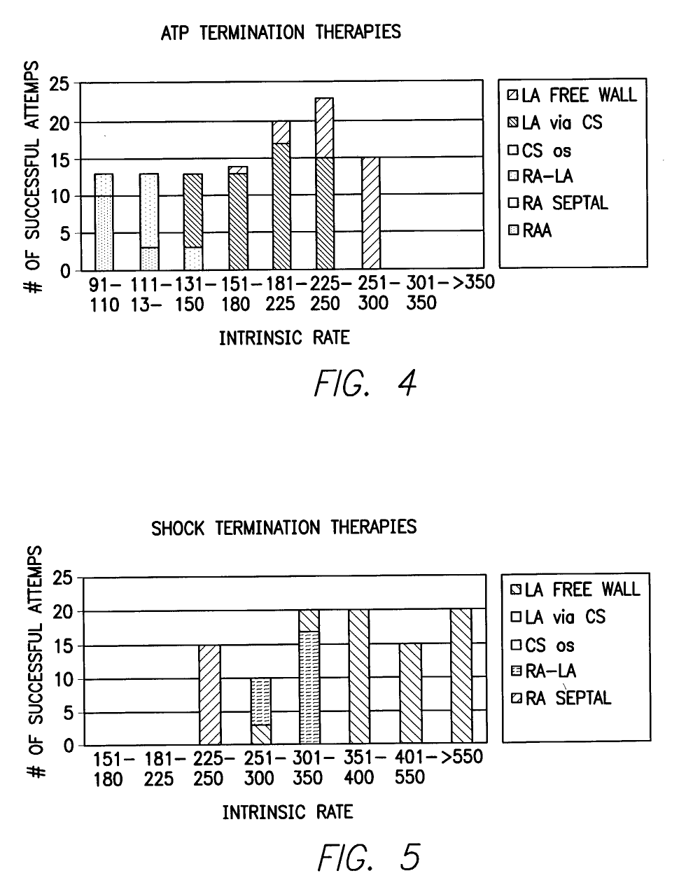

To this end, it will be noted that the device 10 further includes an electrode configuration selector 62 which selects the electrode configuration to be used during the termination of the detected atrial accelerated arrhythmia. The electrode configuration selector 62 maintains in memory 94 a statistical record of the effectiveness of the various electrode configurations versus atrial rate of the atrial arrhythmias. As will be noted in FIG. 4, for example, it shows a statistical record of the number of successful arrhythmia termination attempts for each one of a plurality of different pacing electrode configurations correlated with intrinsic atrial rate. FIG. 5 presents a similar statistical record for atrial defibrillation shock therapy.

It will be noted in FIGS. 4 and 5 that at lower intrinsic atrial rates, pacing therapy is considered most effective. As the atrial rate increases, the pacing therapy electrode selection generally migrates from the right side of the heart or right atrial appendage pacing electrode 22 to the left side of the heart or the left atrial freewall pacing electrode 36. Similarly in FIG. 5, the defibrillation electrode configuration selection generally migrates from the right atrial septal defibrillation coil electrode 28 to the left atrial freewall coil electrode 38. For atrial rates in the mid range for which either pacing or shock therapies are available, pacing therapy is generally more desirable in as much as pacing therapy is not as perceptible to the patient as shock therapy. Also, since atrial tachycardia and fibrillation are not immediately life threatening, there is ample time to attempt termination with pacing therapy before having to turn to the more aggressive shock therapy.

It will also be noted in FIG. 2 that the device 10 further includes a therapy selector 64. The therapy selector determines whether the atrial accelerated arrhythmia should be terminated with pacing therapy or defibrillation shock therapy. Hence, the selection of the therapy to be applied may be made based upon two considerations. A first consideration is the type of therapy to be used based on atrial rate and the second consideration is the electrode configuration for applying the selected type of therapy based both upon the intrinsic rate of the atrial tachyarrhythmia and the selected type of therapy.

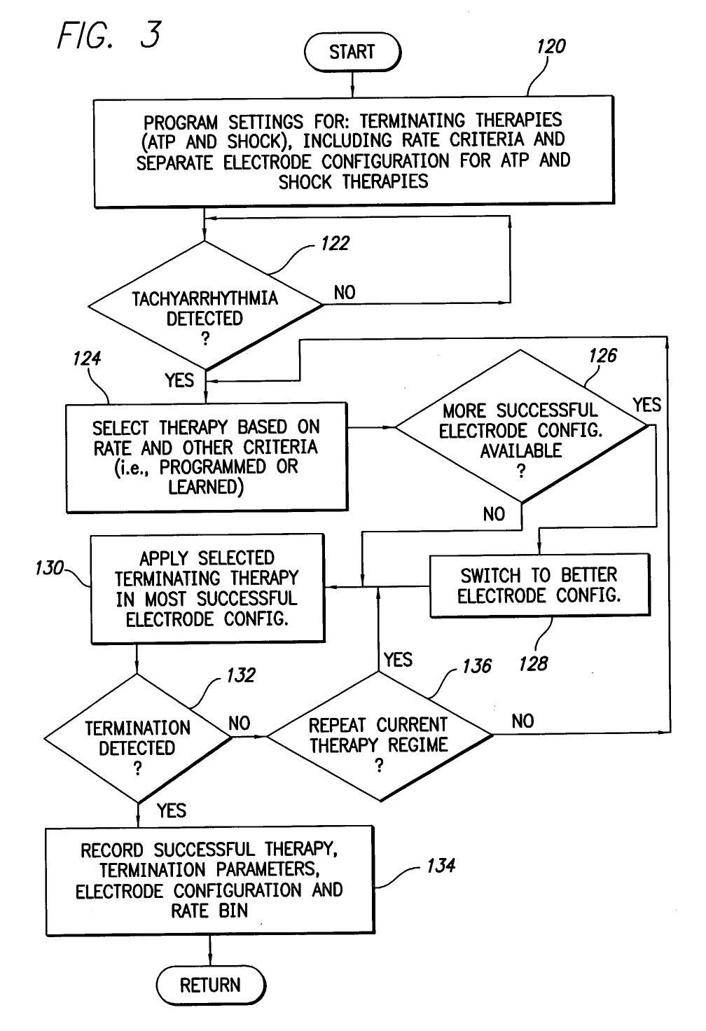

Referring now to FIG. 3, FIG. 3 is a flow chart describing an overview of the operation and novel features implemented in one embodiment of the device 10. In this flow chart, the various algorithmic steps are summarized in individual “blocks”. Such blocks describe specific actions or decisions that must be made or carried out as the algorithm proceeds. Where a microcontroller (or equivalent) is employed, the flow chart presented herein provides the basis for a “control program” that may be used by such a microcontroller (or equivalent) to effectuate the desired control of the stimulation device. Those skilled in the art may readily write such a control program based on the flow chart and other descriptions presented herein.

The process of FIG. 3 initiates in activity block 120 by setting program settings for the termination therapies. The termination therapies, as previously described, may be atrial tachycardia pacing and atrial defibrillation shock therapy. The program settings would include rate criteria and separate electrode configurations for atrial tachycardia pacing and atrial defibrillation shock therapies.

Following activity block 120, the process advances to decision block 122. Here it is determined if an atrial tachyarrhythmia has been detected. If not, the process continuously detects for an atrial tachyarrhythmia. When an atrial tachyarrhythmia is detected, the process then advances to activity block 124 wherein the therapy selector 64 selects the appropriate therapy for terminating the detected atrial tachyarrhythmia. The therapy may be selected based upon the intrinsic atrial rate of the tachyarrhythmia and/or other criteria. Such other criteria may include onset characteristics of the tachyarrhythmia, the degree of organization of the tachyarrhythmia, or the morphology of the atrial activity. As previously mentioned, for lower rate tachyarrhythmias, atrial tachycardia pacing is generally preferred whereas for the higher rate atrial tachyarrhythmias such as atrial fibrillation, more aggressive defibrillation shock therapy may be preferred.

Following activity block 124, the process advances to decision block 126. Here, the electrode configuration selector 62 determines if an electrode configuration is available which would be more successful in terminating the detected atrial accelerated arrhythmia than the electrode configuration currently set for use. In performing decision block 126, the electrode configuration selector 62 makes use of its statistical record of the degree of success for each of the electrode configurations correlated with intrinsic atrial rate.

If a better electrode configuration is available as determined in decision block 126, the process advances to activity block 128 wherein the electrode configuration switch 74 switches the shocking circuit 116 or one of pulse generators 70 and 72, depending on the therapy type selections to the more successful electrode configuration. The process then advances to activity block 130. If a more successful electrode configuration was not available as determined in decision block 126, the process advances directly to activity block 130.

In activity block 130, the selected termination therapy is applied to the selected electrode configuration. After the therapy has been applied, whether pacing therapy or defibrillation shock therapy, the process advances to decision block 132 wherein it is determined if the therapy was successful in terminating the tachyarrhythmia. If the therapy was successful, the process advances to 134 wherein the statistical record maintained by the electrode configuration selector 62 is updated with the appropriate termination parameters of electrode configuration and rate. The process then returns.

If the therapy was unsuccessful at terminating the atrial tachyarrhythmia, the process then advances to decision block 136 wherein it is determined if the current therapy should be repeated. This decision may be performed by the therapy selector 64. The program settings may call for a certain number of repeated atrial tachycardia pacing attempts to be performed before progressing to the more aggressive therapy of defibrillation shock therapy. Also, the program settings may include an indication of the number of defibrillation shock therapies that should be attempted at a given output before increasing the output of the shocking therapy. All of these parameters may be called into scrutiny in performing decision block 136.

If the last used therapy is to be repeated, the process returns to activity block 130. If, however, the current therapy is not to be repeated, the process then returns to activity block 124 for the selection of a new therapy. Here, for example, the output of the defibrillation shocks may be increased, the therapy may be switched from pacing therapy to defibrillation shock therapy, or some other therapy parameters such as pacing rate may be changed. Any time there is a change in the termination therapy, the process advances then to decision block 126 to determine the most successful electrode configuration to be used for application of the new therapy.

While the invention has been described by means of specific embodiments and applications thereof, it is understood that numerous modifications and variations may be made thereto by those skilled in the art without departing from the spirit and scope of the invention. It is therefore to be understood that within the scope of the claims, the invention may be practiced otherwise than as specifically described herein.

What is claimed is:

1. In an implantable cardiac stimulation device, a system for terminating atrial accelerated arrhythmias of a heart by delivering an atrial accelerated arrhythmia termination therapy, the system comprising:
one or more leads;
a plurality of electrodes connected to the one or more leads, wherein the electrodes are configured to define a plurality of electrode configurations to deliver the atrial accelerated arrhythmia termination therapy;
an arrhythmia detector that is operative to detect atrial accelerated arrhythmias of the heart and to determine a measure of at least one predetermined characteristic of the atrial accelerated arrhythmias;
an electrode configuration selector that selects one of the plurality of electrode configurations based upon the measure of at least one predetermined characteristic of the detected atrial accelerated arrhythmia; and
a therapy circuit that provides the atrial accelerated arrhythmia termination therapy to the selected electrode configuration.

2. The system of claim 1 wherein the electrode configuration selector selects a most effective electrode configuration for termination of the detected atrial accelerated arrhythmia from among the plurality of electrode configurations.

3. The system of claim 2 wherein the electrode configuration selector maintains a statistical record correlating electrode configuration atrial accelerated arrhythmia termination effectiveness with measures of the predetermined characteristic.

4. The system of claim 3 wherein the electrode configuration selector updates the statistical record after each atrial accelerated arrhythmia termination.

5. The system of claim 1 wherein the at least one predetermined characteristic is atrial rate.

6. The system of claim 1 further comprising a therapy selector that selects the atrial accelerated arrhythmia termination therapy from among a plurality of atrial accelerated arrhythmia termination therapies based upon the at least one predetermined characteristic.

7. The system of claim 6 wherein the electrode configuration selector selects the one of the plurality of electrode configurations for termination of the detected atrial accelerated arrhythmia based upon the measure of the at least one predetermined characteristic and the atrial accelerated arrhythmia termination therapy selected by the therapy selector.

8. The system of claim 6 wherein the plurality of atrial accelerated arrhythmia therapies include atrial tachycardia pacing and atrial defibrillation.

9. An implantable system for delivering an atrial antitachycardia therapy to terminate an atrial accelerated arrhythmias of a heart, the system comprising:
means for defining a plurality of electrode configurations capable of delivering the atrial antitachycardia therapy;
means for detecting an atrial accelerated arrhythmia of the heart and for determining a measure of at least one predetermined characteristic of the atrial accelerated arrhythmia;
means for selecting one of the plurality of electrode configurations based upon the measure of the at least one predetermined characteristic; and
stimulating means for providing the atrial antiarrhythmia therapy to the selected electrode configuration.

10. The system of claim 9 wherein the selecting means includes means for selecting a most effective electrode configuration for termination of the detected atrial accelerated arrhythmia from among the plurality of electrode configurations.

11. The system of claim 10 wherein the selecting means includes means for maintaining a statistical record correlating electrode configuration atrial accelerated arrhythmia termination effectiveness with measures of the predetermined characteristic.

12. The system of claim 11 wherein the selecting means includes means for updating the statistical record after each atrial accelerated arrhythmia termination.

13. The system of claim 9 wherein the at least one predetermined characteristic is atrial rate.

14. The system of claim 9 further comprising a therapy selecting means for selecting an atrial accelerated arrhythmia termination stimulation therapy from among a plurality of atrial accelerated arrhythmia termination stimulation therapies based upon the at least one predetermined characteristic.

15. The system of claim 14 wherein the selecting means includes means for selecting the one of the plurality of electrode configurations for termination of the detected atrial accelerated arrhythmia based upon the measure of the at least one predetermined characteristic and the atrial accelerated arrhythmia termination stimulation therapy selected by the therapy selecting means.

16. The system of claim 14 wherein the plurality of atrial accelerated arrhythmia stimulation therapies include atrial tachycardia pacing and atrial defibrillation.

17. In an implantable cardiac stimulation system, a method of delivering an atrial antitachycardia therapy to terminate an atrial accelerated arrhythmia of a heart, the method comprising:
providing a plurality of electrode configurations capable of delivering the atrial antitachycardia therapy;
detecting an atrial accelerated arrhythmia and determining a measure of at least one predetermined characteristic of the atrial accelerated arrhythmia;
selecting one of the plurality of electrode configurations based upon the measure of the at least one predetermined characteristic; and
providing the atrial antitachycardia therapy to the selected electrode configuration.

18. The method of claim 17 wherein selecting comprises selecting an optimal electrode configuration for termination of the detected atrial accelerated arrhythmia from among the plurality of electrode configurations.

19. The method of claim 18 further comprising maintaining a statistical record correlating electrode configuration atrial accelerated arrhythmia termination effectiveness with measures of the predetermined characteristic.

20. The method of claim 19 wherein maintaining comprises updating the statistical record after each atrial accelerated arrhythmia termination.

21. The method of claim 17 wherein the at least one predetermined characteristic is atrial rate.

22. The method of claim 17 further comprising selecting an atrial accelerated arrhythmia termination stimulation therapy from among a plurality of atrial accelerated arrhythmia termination stimulation therapies based upon the at least one predetermined characteristic.

23. The method of claim 22 wherein selecting comprises selecting the one of the plurality of electrode configurations for termination of the detected atrial accelerated arrhythmia based upon the measure of the at least one predetermined characteristic and the selected atrial accelerated arrhythmia termination stimulation therapy.

24. The method of claim 22 wherein the plurality of atrial accelerated arrhythmia stimulation therapies include atrial tachycardia pacing and atrial defibrillation.
